SEQUENCE LISTING (15 sequences)

SEQ ID NO: 1 - DNA, 7196 bp, artificial sequence
  chemically synthesized
gtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgcctttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttcattaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccccatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagttgcgtgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtcgatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaaccgctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggcccggatccaagcttacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgaagcttagatctcggctagctagtacttaattaacctaaggcactacgtcttctaaacctgccaagcgtgtgcgtgtacgtgccaggaagtaatat

SEQ ID NO: 2 - DNA, 8230 bp, artificial sequence
  chemically synthesized
gtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgcctttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttcattaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccccatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagttgcgtgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtcgatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaaccgctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggcccggatccaagcttacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgaagcttagatctcggctagcgtatacggatcgatcctgcaggtcgactctagacaggtaagtggcgtttctcggggagccagctgcgtccgctgtcgtgctgtcggtgtagtactagcaagcgttaagtccccatctggctgcggcctaccgaagagtggtcttcacgtcacacgctgtcccacgcacgtggttggtttggtcgcttctggttactgactactaagcagccttttcttttttcctttcaggttctagacgccaccatgggcgtgaaggtgctgttcgccctgatctgtatcgccgtggccgaggccaagcccaccgagaacaacgaggacttcaacatcgtggccgtggccagcaacttcgccaccacagacctggacgccgacagaggcaagctgcccggcaagaaactgcccctggaagtgctgaaagagatggaagccaacgccagaaaggccggctgcaccagaggctgcctgatctgcctgagccacatcaagtgcacccccaagatgaagaagttcatccccggcagatgccacacctacgagggcgacaaagagagcgcccagggcggcatcggcgaggccatcgtggacatccccgagatccccggcttcaaggacctggaacccatggaacagtttatcgcccaggtggacctgtgcgtggactgcaccaccggctgtctgaagggcctggccaacgtgcagtgcagcgacctgctgaagaagtggctgccccagagatgcgccaccttcgccagcaagatccagggccaggtggacaagatcaagggcgctggcggcgactgatgagcggccgcctcgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcttaattaacctaaggcactacgtcttctaaacctgccaagcgtgtgcgtgtacgtgccaggaagtaatat

SEQ ID NO: 3 - DNA, 8983 bp, artificial sequence
  chemically synthesized
gtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgcctttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttcattaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccccatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagttgcgtgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtcgatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaaccgctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggcccggatccaagcttacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgaagcttagatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagctagcgtatacggatcgatcctgcaggtcgactctagacaggtaagtggcgtttctcggggagccagctgcgtccgctgtcgtgctgtcggtgtagtactagcaagcgttaagtccccatctggctgcggcctaccgaagagtggtcttcacgtcacacgctgtcccacgcacgtggttggtttggtcgcttctggttactgactactaagcagccttttcttttttcctttcaggttctagacgccaccatgggcgtgaaggtgctgttcgccctgatctgtatcgccgtggccgaggccaagcccaccgagaacaacgaggacttcaacatcgtggccgtggccagcaacttcgccaccacagacctggacgccgacagaggcaagctgcccggcaagaaactgcccctggaagtgctgaaagagatggaagccaacgccagaaaggccggctgcaccagaggctgcctgatctgcctgagccacatcaagtgcacccccaagatgaagaagttcatccccggcagatgccacacctacgagggcgacaaagagagcgcccagggcggcatcggcgaggccatcgtggacatccccgagatccccggcttcaaggacctggaacccatggaacagtttatcgcccaggtggacctgtgcgtggactgcaccaccggctgtctgaagggcctggccaacgtgcagtgcagcgacctgctgaagaagtggctgccccagagatgcgccaccttcgccagcaagatccagggccaggtggacaagatcaagggcgctggcggcgactgatgagcggccgcctcgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcttaattaacctaaggcactacgtcttctaaacctgccaagcgtgtgcgtgtacgtgccaggaagtaatat

SEQ ID NO: 4 - DNA, 11319 bp, artificial sequence
  chemically synthesized
attaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagtgcgttgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtgcatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaacggctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtcccgacctcgtgaaataaaagtgcagaaaacaaacccaggcgatcacagcagcagccgccgcggcagcagcaccaacagcaggaggagcaggaggagccggaggaggaggaggaggaggaggcaaagttagagttggggctggcgctccggagttgctgggctcagcgcagctcccattcattaaggaaccagctgcggaggaaggtggccgagcgcccgcgctgcccactcgctcgctcgcgcactcagacgcgcgccacaacagcgcgccccaagctgcgcagctctgcaaaagtttctgctcgggatctggctctcttccccttggactttagaacgatttagggttgacagaggaaagcagaggcgcgcaggaggagcagaaaacaccaccttctgcagttggaggcaggcagccccggctgcactctagccgccgcgcccggagccggggccgacccgccactatccgcagcagcctcggccaggaggcgacccgggcgcctgggtgtgtggctgctgttgcgggacgtcttcgcggggcgggaggctcgcgccgcagccagcgccatggccacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattctagagcggccgcaagcttaattaacgtctcgcactacgtcttctaaacctgccaagcgtgtgcgtgtacgtgccaggaagtaatatgtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgccagatctgatatctctagagtcgacccatgggggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactagtgaattcagatcttttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttc

SEQ ID NO: 5 - DNA, 11541 bp, artificial sequence
  chemically synthesized
attaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagtgcgttgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtgcatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaacggctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtcccgacctcgtgaaataaaagtgcagaaaacaaacccaggcgatcacagcagcagccgccgcggcagcagcaccaacagcaggaggagcaggaggagccggaggaggaggaggaggaggaggcaaagttagagttggggctggcgctccggagttgctgggctcagcgcagctcccattcattaaggaaccagctgcggaggaaggtggccgagcgcccgcgctgcccactcgctcgctcgcgcactcagacgcgcgccacaacagcgcgccccaagctgcgcagctctgcaaaagtttctgctcgggatctggctctcttccccttggactttagaacgatttagggttgacagaggaaagcagaggcgcgcaggaggagcagaaaacaccaccttctgcagttggaggcaggcagccccggctgcactctagccgccgcgcccggagccggggccgacccgccactatccgcagcagcctcggccaggaggcgacccgggcgcctgggtgtgtggctgctgttgcgggacgtcttcgcggggcgggaggctcgcgccgcagccagcgccatggccacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattctagagcggccgcctcgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcttaattaacgtctcgcactacgtcttctaaacctgccaagcgtgtgcgtgtacgtgccaggaagtaatatgtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgccagatctgatatctctagagtcgacccatgggggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactagtgaattcagatcttttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttc

SEQ ID NO: 6 - DNA, 7320 bp, artificial sequence
  chemically synthesized
attaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagtgcgttgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtgcatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaacggctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtcccgacctcgtgaaataaaagtgcagaaaacaaacccaggcgatcacagcagcagccgccgcggcagcagcaccaacagcaggaggagcaggaggagccggaggaggaggaggaggaggaggcaaagttagagttggggctggcgctccggagttgctgggctcagcgcagctcccattcattaaggaaccagctgcggaggaaggtggccgagcgcccgcgctgcccactcgctcgctcgcgcactcagacgcgcgccacaacagcgcgccccaagctgcgcagctctgcaaaagtttctgctcgggatctggctctcttccccttggactttagaacgatttagggttgacagaggaaagcagaggcgcgcaggaggagcagaaaacaccaccttctgcagttggaggcaggcagccccggctgcactctagccgccgcgcccggagccggggccgacccgccactatccgcagcagcctcggccaggaggcgacccgggcgcctgggtgtgtggctgctgttgcgggacgtcttcgcggggcgggaggctcgcgccgcagccagcgccatggccacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattctagagcggccgcaagcttaattaacgtctcgcactacgtcttctaaacctgccaagcgtgtgcgtgtacgtgccaggaagtaatatgtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgccagatctgatatctctagagtcgacccatgggggcccgccccaactggggtaacctttgggctccccgggcgcgactagtgaattcagatcttttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttc

SEQ ID NO: 7 - DNA, 7542 bp, artificial sequence
  chemically synthesized
attaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagtgcgttgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtgcatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaacggctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtcccgacctcgtgaaataaaagtgcagaaaacaaacccaggcgatcacagcagcagccgccgcggcagcagcaccaacagcaggaggagcaggaggagccggaggaggaggaggaggaggaggcaaagttagagttggggctggcgctccggagttgctgggctcagcgcagctcccattcattaaggaaccagctgcggaggaaggtggccgagcgcccgcgctgcccactcgctcgctcgcgcactcagacgcgcgccacaacagcgcgccccaagctgcgcagctctgcaaaagtttctgctcgggatctggctctcttccccttggactttagaacgatttagggttgacagaggaaagcagaggcgcgcaggaggagcagaaaacaccaccttctgcagttggaggcaggcagccccggctgcactctagccgccgcgcccggagccggggccgacccgccactatccgcagcagcctcggccaggaggcgacccgggcgcctgggtgtgtggctgctgttgcgggacgtcttcgcggggcgggaggctcgcgccgcagccagcgccatggccacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattctagagcggccgcctcgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcttaattaacgtctcgcactacgtcttctaaacctgccaagcgtgtgcgtgtacgtgccaggaagtaatatgtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgccagatctgatatctctagagtcgacccatgggggcccgccccaactggggtaacctttgggctccccgggcgcgactagtgaattcagatcttttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttc

SEQ ID NO: 8 - DNA, 34 bp, artificial sequence
  chemically synthesized
cgcgaggtcccgacctcgtgaaataaaagtgcag

SEQ ID NO: 9 - DNA, 66 bp, artificial sequence
  chemically synthesized
agtcagtgcgagacgttaattaagcttgcggccgcgtatacaagcttccatggcgctggctgcggc

SEQ ID NO: 10 - DNA, 13024 bp, artificial Sequence
  chemically synthesized
attaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagttgcgtgcaggacaaaatcatagaccactatgaagccacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaagcaccggtgaaacagactttgaattttgaccttctcaagttggcgggagacgtggagtccaaccctgggcccatgcagacaccgaaggaaaccctttcggaacgtttaagtgcgttgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtgcatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaacggctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtccccgcctttacagtagggcctaccaacaagtgtcagtggctaaccctgagtttcttacacgtccatcctctttaattacatatgacaacccggcctttgagcctgtggacactacattaacatttgatcctcgtagtgatgttcctgattcagattttatggatattatccgtctacataggcctgctttaacatccaggcgtgggactgttcgctttagtagattaggtcaacgggcaactatgtttacccgcagcggtacacaaataggtgctagggttcacttttatcatgatataagtcctattgcaccttccccagaatatattgaactgcagcctttagtatctgccacggaggacaatgacttgtttgatatatatgcagatgacatggaccctgcagtgcctgtaccatcgcgttctactacctcctttgcattttttaaatattcgcccactatatcttctgcctcttcctatagtaatgtaacggtccctttaacctcctcttgggatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctagtgacaataccgtatatcttccacctccttctgtggcaagagttgtaaataccgatgattatgtgactcgcacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcgtgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcgcagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttccccccccgccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcaggctggattgcgtcgcaagcccaccataggccctcgcaaacgttctgctccatctgccactacgtcttctaaacctgccaagcgtgtgcgtgtacgtgccaggaagtaatatgtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgccagatctgatatctctagagtcgacccatgggggcccgccccaactggggtaacctttgagttctctcagttgggggtaatcagcatcatgatgtggtaccacatcatgatgctgattataagaatgcggccgccacactctagtggatctcgagttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgtagatgacatggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgctcgaggggggccaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggtcgagactcagcggccgcggtgccagggcgtgcccttgggctccccgggcgcgactagtgaattcagatcttttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttc

SEQ ID NO: 11 - DNA, 9025 bp, artificial sequence
  chemically synthesized
attaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagttgcgtgcaggacaaaatcatagaccactatgaagccacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaagcaccggtgaaacagactttgaattttgaccttctcaagttggcgggagacgtggagtccaaccctgggcccatgcagacaccgaaggaaaccctttcggaacgtttaagtgcgttgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtgcatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaacggctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtccccgcctttacagtagggcctaccaacaagtgtcagtggctaaccctgagtttcttacacgtccatcctctttaattacatatgacaacccggcctttgagcctgtggacactacattaacatttgatcctcgtagtgatgttcctgattcagattttatggatattatccgtctacataggcctgctttaacatccaggcgtgggactgttcgctttagtagattaggtcaacgggcaactatgtttacccgcagcggtacacaaataggtgctagggttcacttttatcatgatataagtcctattgcaccttccccagaatatattgaactgcagcctttagtatctgccacggaggacaatgacttgtttgatatatatgcagatgacatggaccctgcagtgcctgtaccatcgcgttctactacctcctttgcattttttaaatattcgcccactatatcttctgcctcttcctatagtaatgtaacggtccctttaacctcctcttgggatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctagtgacaataccgtatatcttccacctccttctgtggcaagagttgtaaataccgatgattatgtgactcgcacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcgtgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcgcagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttccccccccgccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcaggctggattgcgtcgcaagcccaccataggccctcgcaaacgttctgctccatctgccactacgtcttctaaacctgccaagcgtgtgcgtgtacgtgccaggaagtaatatgtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgccagatctgatatctctagagtcgacccatgggggcccgccccaactggggtaacctttgggctccccgggcgcgactagtgaattcagatcttttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttc

SEQ ID NO: 12 - DNA, 73 bp, artificial sequence
  chemically synthesized
tcggaacgtttaagttgcgtgcaggacaaaatcatagaccactatgaagccacttcgaaagtttatgatccag

SEQ ID NO: 13 - DNA, 90 bp, artificial sequence
  chemically synthesized
ttggactccacgtctcccgccaacttgagaaggtcaaaattcaaagtctgtttcaccggtgcttgttcatttttgagaactcgctcaacg

SEQ ID NO: 14 - DNA, 48 bp, artificial sequence
  chemically synthesized
ggagacgtggagtccaaccctgggcccatgcagacaccgaaggaaacc

SEQ ID NO: 15 - DNA, 21 bp, artificial sequence
  chemically synthesized
cacagtgtccaggtcgtgtag

(19) United States
(12) Patent Application Publication
(10) Pub. No.: US 2013/0150262 A1
(43) Pub. Date: Jun. 13, 2013

(54) Method and Kit for Identifying Compounds Capable of Inhibiting Human Papilloma Virus Replication

(71) Applicant: Icosagen Cell Factory OU, Tartu (EE)

(72) Inventors: Mart Ustav, Tartu (EE); Ene Ustav, Tartu (EE); Jelizaveta Geimanen, Tartu (EE); Regina Pipits, Tartu (EE); Helen Isok-Paas, Tallinn (EE); Tormi Reinson, Tartu (EE); Mart Ustav, Jr., Tartu (EE); Triin Laos, Parnu (EE); Marit Orav, Harjumaa (EE); Kristiina Salk, Tallinn (EE); Andres Mannik, Tartu (EE); Anu Remm, Tartu (EE)

(73) Assignee: ICOSAGEN CELL FACTORY OU, Tartu (EE)

(21) Appl. No.: 13/680,618

(22) Filed: Nov. 19, 2012

(30) Foreign Application Priority Data
May 19, 2010 (EE) PCT/EE2010/000010

(51) Int. Cl. C12Q 1/70

(52) U.S. Cl. CPC C12Q 1/708
USPC 506/13; 435/5; 435/366; 435/320.1

(57) ABSTRACT

This invention provides a method, kit and an in vitro system for identifying compounds capable of inhibiting Human Papilloma Virus replication at all the stages of viral replication cycle. The method, kit and in vitro system is applicable to all types of Human Papilloma Virus. The method enables high throughput screening of compounds inhibiting HPV replication in one or more phases of the cycle.

### PRIORITY

This application is a continuation in part application of International Application Number PCT/EE2010/000010 filed on May 19, 2010 which is incorporated herein by reference in its entirety.

### SEQUENCE LISTING

This application contains sequence listing.

### TECHNICAL FIELD OF THE INVENTION

The present invention relates to the fields of virology, cell biology, cell culturing, and drug development. More particularly the invention provides a method for screening for anti-HPV substances and a kit for screening for anti-HPV substances. The invention also provides plasmids for transfecting cell lines and cell lines capable of supporting all replication phases of Human Papilloma Virus.

### BACKGROUND OF THE INVENTION

The continuous interest to study the human papillomaviruses (HPV) has been generated from their association with specific human cancers. HPV infects basal proliferating cells of the epithelium and induces the formation of benign tumors. In some cases this infection may lead to progression and formation of malignant carcinomas. The complete papillomavirus virion constitutes a protein coat (capsid) surrounding a circular, double-stranded DNA organized into coding and non-coding regions. Eight early (E1-E8) open reading frames (ORFs) and two late (L1, L2) ORFs have been identified in the coding region of papillomaviruses. The early ORFs encode proteins involved in viral DNA replication during the establishment, in continuous maintenance state and late amplification (E1 and E2), in regulation of viral gene expression and chromosome tethering (E2), virus assembly (E4), immortalisation and transformation (E6 and E7/high-risk HPVs only). Late ORFs are activated only after cell differentiation and encode viral capsid proteins (L1 and L2). In the noncoding Upstream Regulatory Region (URR) the promoters, enhancer and other regulatory elements in addition to the replication origin are located.

The current view divides the papillomaviral life cycle into three stages. First, following initial entry into the cell nucleus in the basal layer of the epithelium, where the apparatus necessary for replication exists, the PV genome is amplified, viral DNA is synthesized faster than chromosomal DNA, the copy number raises (up to 50-300 copies per cell) (for review, see Kadaja M, Silla T, Ustav E, Ustav M. Papillomavirus DNA replication—from initiation to genomic instability. Virology. 2009 Feb. 20; 384(2):360-8.). The second stage represents stable replication of HPV DNA in S-phase synchronized with chromosomal replication and maintenance of viral DNA as extrachromosomal multicopy nuclear episomes as a result of segregation/partitioning of the viral genome into the daughter cells.

At this stage only early genes are expressed and neither the synthesis of capsid proteins L1 and L2 nor virion assembly occurs. Early gene products provide transforming proteins that ensure clonal expansion of infected cells. If infected cells detach from the basal membrane and reach upper layers of the skin or mucosa, they stop dividing and start differentiation (keratinisation). It triggers onset of the third step, vegetative viral DNA replication during which a) viral DNA amplification is initiated again, and then b) late proteins are synthesized and viral particles assembled (for review, see Kadaja M, Silla T, Ustav E, Ustav M. Papillomavirus DNA replication—from initiation to genomic instability. Virology. 2009 Feb. 20; 384(2):360-8.).

Modelling of these replication stages in cells has been problematic in the case of human papillomaviruses. Most of the tissue culture cells do not support any mode of HPV genomic replication. Attempts to get viral genomic DNA replication going from transfected plasmids of β-papillomavirus types has completely failed in any keratinocyte cell lines or primary keratinocytes. Also, it has been difficult to generate reproducible human cell lines that carry stable HPV replicating genomes, especially that of the “low risk”-HPV types. The stable replication of HPV episomes has been accomplished just by a handful of laboratories. The episomal state has been shown to be allowed only in the presence of feeders or in conditions of raft cultures. W12, a frequently used HPV-16 cell line, has originated from a patient sample, but while cultivating W12 cells in monolayer, integration events have been shown to take place instead of maintenance of the episomal state of the viral genome.

Nevertheless, the replication of HPV replication origin containing plasmids can be demonstrated in many different cell lines of different species in case the production of E1 and E2 proteins is provided from heterologous expression vectors. The main factor which restricts the replication to certain epithelial cells is therefore the availability of coordinated expression of cellular transcription factors for the transcription of the mRNAs for viral proteins.

The vaccines targeting HPV-16 and HPV-18 or HPV-6b, HPV-11, HPV-16 and HPV-18 have been developed and are becoming increasingly available in many countries. It should be considered as a great achievement in fighting against cervical cancers. However, it is not sufficient, because the vaccines target at best only for four subtypes of hundreds of papillomaviruses, including “high risk”-type of mucosal or cutaneous skin papillomaviruses. Additionally, it has been shown convincingly that HPV-16 and HPV-18 are prevalent viruses found in cervical carcinomas in developed countries. According to the molecular epidemiological analysis of the spread of the virus in developing countries, like in Sub-Saharan regions of Africa, other virus isolates like HPV-52 and HPV-35 are prevalent.

There is an urgent need for the small-molecule drugs, which can be used to block effectively the replication of the papillomavirus genome, therefore lowering the viral load per cell and avoiding the generation of viral particles and therefore the spread of the virus. Furthermore, there is a need for small-molecule drugs, which could be used in various stages of virus infection to stop the viral replication at that specific stage. However, this objective has been difficult to achieve due to the lack of an effective cellular system for screening for drug candidates. This cellular system should be compatible with the high-throughput and high-content format of the screening of the drug candidates and allow identifying the active substances in reproducible and cost-effective format. Furthermore such cellular system should allow detection of compounds inhibiting any of the replications phases of all types of HPV-viruses. Animal xenograft models have been described previously by J. Duan, WO0040082 (A reproducible xenograft animal model for hosting and propagating human papillomavirus (HPV)), as well as primary keratinocytes are applied for hosting the viral genome by Kreider et al. 1993 and 1998, (U.S. Pat. No. 5,541,058, In vitro assay system for testing the effectiveness of anti-papilloma viral agents; U.S. Pat. No. 6,200,745, Vitro assay system using a human cell line for testing the effectiveness of anti-papilloma viral agents). However, these methods do not allow high-throughput screening for drug candidates, and a simpler and more convenient method is necessarily required. Our group has previously discovered the ability of human osteosarcoma cell line U2OS to support the in vitro cultivation of HPV (K. Salk, 2009 Studies on the mechanisms of the DNA replication of high- and low-risk human papillomavirus in different cell lines. MSc thesis /in Estonian/; University of Tartu Press). However, maintenance of episomal HPV by itself is not sufficient for a high-throughput screening assay to identify possible HPV replication inhibitors.

### SUMMARY OF THE INVENTION

This invention provides solutions to the above described shortcomings of current technology and others.

Accordingly it is an object of this invention to provide a cellular system supporting all phases of HPV DNA replication to allow determination of inhibitory effects of drug candidates on various phases of HPV DNA replication.

It is another object of this invention to provide a method to screen for factors inhibiting the HPV DNA replication at all different replication phases of HPV life cycle by detecting a product of a reporter gene or a reaction product of a protein encoded by a reporter gene enabling.

Another object of this invention is to provide a method to screen for factors inhibiting DNA replication of all types of human papilloma viruses, including high-risk, low-risk and cutaneous HPV on various phases of HPV DNA replication.

Another object of this invention is to provide extrachromosomally maintainable plasmids carrying HPV DNA sequences for transfection of cell lines.

Yet another object of this invention is to provide cell lines supporting all phases of HPV DNA replication for use in high-throughput screening of HPV replication inhibitors.

Another object of this invention is to provide an in vitro system to screen compounds capable of inhibiting initial replication of HPV DNA for use as vaccines.

Yet another object of this invention is to provide an in vitro system to screen compounds capable of inhibiting stable maintenance of HPV DNA replication for use as vaccines and cure.

An even further object of this invention is to provide an in vitro system to screen compounds capable of inhibiting vegetative amplificational replication phase of HPV DNA to prevent or cure viral infections in nondividing cells in upper layers of skin.

A yet another object of this invention is to identify compounds capable of inhibiting HPV DNA replication either in initial replication, stable maintenance, or vegetative amplification phase of all types of HPV.

Another object of this invention is candidate compounds for treating and curing infections and conditions caused by any type of HPV where the compound is indentified by the method of this invention.

### DISCLOSURE OF THE INVENTION

### Definitions

Initial replication or transient replication refers to HPV DNA replication at establishment of the infection.

Stable maintenance or latent maintenance refers to the latent stage of viral replication cycle where viral DNA is stably maintained at an almost constant copy number in dividing host cells.

Vegetative amplificational replication or late amplificational replication refers to exponential viral DNA amplification when epithelial cells detach the basement membrane.

The present invention provides a method for identifying compounds capable of inhibiting Human Papillomavirus (HPV) DNA replication as well as plasmids for transfecting cells, cell lines capable of supporting all phases of HPV DNA replication and a kit for identifying the compounds capable of inhibiting HPV DNA replication.

The present invention provides a method and a system, wherein HPV genomic or subgenomic DNA is inserted into a cell line, and wherein all the phases of HPV DNA replication are supported, and further the influence of a compound on the HPV DNA replication is determined. The U2OS cell line was identified as a feasible host cell line to support HPV DNA replication. Now, according to the present invention, U2OS cells are identified as a suitable host for the propagation of genomes of all types of mucosal and cutaneous tissue specific HPVs and for the HPV genome-related constructs. It is also demonstrated that amplificational replication of the HPV genome, resembling amplification in the vegetative phase of the viral life-cycle occurs, when HPV positive U2OScell clones are maintained in high density for extended periods with regular media for at least 4 to 12 days.

Thus, a method is provided, wherein the quantitative detection of replicated HPV DNA or, more preferably, detection of a product of a reporter gene, a fusion protein including a reporter gene, or a reaction product of a protein encoded by a reporter gene enables screening for factors inhibiting the HPV DNA replication at all different replication phases of HPV life cycle: a) the initial amplificational replication demonstrated by the transient replication assay; b) the stable HPV DNA replication, synchronous with cellular DNA replication, demonstrated by the analysis of low to high HPV-content subclones; and c) the amplificational replication resembling vegetative phase of the viral DNA replication. This kind of novel system and method can be widely used in pharmacological research and high through-put screening for new potential drug candidates for prevention or therapy of infections by various subtypes of HPVs.

A preferred embodiment of this invention is a method for identifying compounds capable of inhibiting HPV DNA of all types of HPV at intial replication, stable maintenance or vegetative amplification phase of replication comprising the following steps:
a. HPV DNA with complete or partial sequence enabling the transient, stable and vegetative replication steps of HPV DNA is introduced into a cell line enabling the transient, stable and vegetative replication of HPV DNA in these cells;
b. cell bank collections of stable subclones carrying extrachromosomal HPV DNA with different copy numbers per cell are generated;
c. a chosen cell subclone (for HPV type or for copy number variations) is cultivated as a disperse monolayer culture of dividing cells and/or the chosen cell subclone is cultivated as a monolayer of dense culture;
d. the compound under investigation is deposited on the monolayer of the chosen subclone carrying the HPV DNA;
e. the presence or absence of the inhibitory effect of the compound on viral DNA maintenance and/or amplification in the cells is assessed;
f. if inhibitory effect on HPV DNA replication of a certain concentration of a compound is observed, the compound is identified as a candidate for HPV DNA replication inhibitor.

The presence or absence of the inhibitory effect is detected as is described below.

According to one preferred embodiment, the invention provides a method for identifying compounds capable of inhibiting HPV DNA latent replication, which comprises the following steps:
a. plasmid with complete or partial sequence of HPV DNA carrying all viral cis-sequences and trans-factors ensuring all steps of viral replication cycles, which may also encompass a sequence of a reporter gene, is introduced into human osteosarcoma cell line U2OS using methods like, but not limited to, electroporation or chemical transfection methods known in the art;
b. the clones of U2OS cell lines that carry extrachromosomally replicating HPV plasmids are isolated using selection markers providing resistance to the antibiotics like G418 or puromycin, or other selection markers known in the art;
c. the identified cell clones carrying different HPV copies per cell are grown, the stability is determined and cell banks of these cell clones are generated;
d. the cells of the subclone selected for identification of HPV latent replication inhibitors are seeded at low density into 96 or 384 well plates, and cells are cultivated for a short period of time until the cells establish about 40% confulency maintaining the HPV DNA replication in the latent phase;
e. subsequently, the compound under investigation is deposited on the cell clone monolayer culture before confluency to identify inhibitors of latent replication;
f. the increase or lack of increase of the HPV copy number in the cells is determined by direct quantitative or semiquantitative measurement of the amount of viral DNA or by measurement of the amounts of the products of the reporter genes inserted into the HPV plasmid;
g. the compound is identified as a candidate for an inhibitor of HPV DNA latent replication if inhibitory effect on HPV DNA stable replication of a certain concentration of the compound is observed.

In an another preferred embodiment, the invention provides a method for identifying compounds capable of inhibiting induced HPV DNA vegetative amplificational replication, which comprises the following steps:
a. plasmid with complete or partial sequence of HPV DNA carrying all viral cis-sequences and trans-factors ensuring all steps of viral DNA replication cycles, which may also encompass a sequence of a reporter gene, is introduced into human osteosarcoma cell line U2OS using methods like, but not limited to, electroporation or chemical transfection methods known in the art;
b. the clones of U2OS cell lines that carry extrachromosomally replicating HPV plasmids are isolated using selection markers providing resistance to the antibiotics like G418 or puromycin, or other selection markers known in the art;
c. the identified cell clones carrying different HPV copies per cell are characterized, their stability determined, amplification quantities measured and cell banks of these cell clones are generated;
d. the cells of the subclone selected for identification of vegetative amplificational replication are seeded into 96 or 384 well plates and let grow at confluency by additional feedings for at least 4 to 12 days for the launch of the exponential amplificational replication phase with increased copy number of the replicated episomal DNA per cell;
e. subsequently, the compound under investigation, the potential drug candidate, is added to the growth medium of the cultivation vessel of the U2OS cell clone monolayers at confluency to identify inhibitors of vegetative amplificational replication;
f. the increase or the lack of increase of the HPV copy number in the cells is determined by direct quantitative or semiquantitative measurement of the amount of viral DNA or by measurement of the amount of the products of the reporter genes inserted into the HPV plasmid;
g. the compound is identified as a candidate for an inhibitor of HPV DNA vegetative amplificational replication if inhibitory effect on HPV DNA replication of a certain concentration of the compound is observed.

According to the present invention launch of vegetative amplification of step d above is achieved with high risk HPV, with low risk HPV and even with cutaneous beta-papilloma viruses.

The inhibitory effect can be determined by any methods known in the art, which enables quantitative detection of the extrachromosomal (plasmid) DNA. However, most preferable methods comprise, but are not limited to, inserting nucleic acid sequences, which encode a reporter gene, to the episomally replicating construct. These reporter genes may encode any directly detectable and measurable proteins known in the art, or proteins catalyzing a reaction, product of which can be measured quantitatively or semiquantitatively, e.g. by visual observation with a microscope. The measurable product may remain inside the cell or may be excreted into the media. Examples of such reporter genes comprise, but are not limited to, dGFP, luciferase, secreted alkaline phosphatase, Gaussia luciferase, Renilla luciferase, dGFP-Luciferase fusion gene. Preferably, the nucleic acid sequence of the reporter gene is inserted to the region of HPV genome, which encodes for the L genes.

Most preferably the nucleic acid sequence of the reporter gene substitutes the L1 or L2 genes or both of them in the HPV genome. According to one preferred embodiment the reporter gene sequences are inserted in E2 ORF after E1 coding sequence. The subclones provided for selection from the generated cell banks are chosen from the ones carrying the variety of copy numbers ranging from low to high copy numbers of HPV plasmid per cell.

The subtypes of HPV provided in the present invention comprise, but are not limited to, HPV-18, HPV-16, HPV-6b, HPV-11, HPV-5 and HPV-8. These subtypes belong to mucosal high-risk, low-risk and cutaneous type of HPV subgroups, thus providing previously undescribed means for detecting substances capable for inhibiting the DNA replication of low-risk and skin-type of HPVs. The latent phase of HPV DNA replication provided in the invention, models the viral DNA replication process occurring in the dividing cells at the basal and suprabasal layer of the skin, infected by HPV. The vegetative amplificational replication phase of HPV replication provided in the invention models the viral DNA replication process occurring in nature in nondividing cells in the upper layers of the skin.

Moreover, the present invention provides a kit for identifying compounds capable of inhibiting HPV DNA initial, stable and amplificational replication. This kit comprises at least: human osteosarcoma cell line U2OS, or another cell line enabling the stable replication of HPV DNA; an episomally maintainable construct with complete or partial sequences of HPV DNA with L1 or L2 genes or both substituted with the reporter genes, or alternatively the reporter genes being inserted in E2 ORF before E1 coding sequence, for introduction into the cell line; a compound or a library of compounds to be screened for anti-HPV activity; and a means for assessing transcriptional activity of HPV DNA in the cells.

Hereby, experimental data is provided to illustrate the ability of U2OS cell line to support HPV DNA replication at establishment, at latent maintenance phase as well as the unexpected phenomena of the induction of exponential viral DNA amplification mimicking the vegetative phase of the infection. The data is provided by way of examples and the scope of the invention is presented in the claims.

### SHORT DESCRIPTION OF THE FIGURES

FIG. 1-FIG. 4. Transient DNA replication assays of mucosal high-risk, low-risk and cutaneous type of HPV subgroups.

U2OS cells were transfected with HPV-16 genome (FIG. 1), with HPV-6b, HPV-11, HPV-18 genomes (FIG. 2); with HPV-5 and HPV-8 genomes (FIG. 3 and FIG. 4) and short term replication assay was performed.

Prior to transfection, the HPV DNAs were cleaved out from the vector backbone: HPV-18 genome from pBR322 vector with EcoRI; HPV-6b from pBR322 with BamHI; HPV-16 and HPV-11 genomes from pUC19 with BamHI; HPV-8 DNA from pUC9 vector with BamHI; HPV-5 from pBR322 with Sad. Linear HPV fragments (ca 8 kb) were religated at low DNA concentrations (5 μg/ml) for 16 hrs at 4° C.

FIG. 1. Detection of dose response of the introduced mucosal type of HR-HPV16 reporter plasmid: 1, 2, and 5 μg of religated circular plasmid DNA of the HPV-16 genome was introduced into U2OS cells. Low-molecular-weight DNA was extracted 24, 48, 72, and 96 hrs post transfection by Hirt lysis method and restriction analysis was performed using linearizing enzyme BamHI and bacterial methylation sensitive DpnI. For Southern blot hybridization the full length HPV-16 specific probe was used. The intensity of the linear 8 kb band increased in time (indicated by arrow), which is considered as the indication of replication of viral genome in these cells. Replication signals increased also concentration dependently.

FIG. 2. Establishment of DNA replication from the LR-HPV-6b, LR-HPV-11 and HR-HPV-18. Religated circular plasmid DNAs of HPV-6b, HPV-11 and HPV-18 genomes (5 μg) were introduced into U2OS cells. The samples of Hirt lysis were digested with appropriate linearizing enzyme (look at markers) additionally to Dpnl, and the replicated HPV DNA signals were detected by Southern blotting with radiolabelled HPV genome-specific probes. The ca 8 kb linear DpnI-resistant replication signals, which are increasing in time, are shown in case of all three investigated papillomavirus types.

FIG. 3. Establishment of DNA replication from the cutaneous type of HPV-5 genome. The religated circular plasmid DNA of HPV-5 genome was titrated (2, 5, 10 μg) into U2OS cells and Hirt lysis samples (episomal DNA, treated with SacI/DpnI) were loaded and viral DNA amplification was detected 24, 48, 72, 96 hrs post transfection by Southern blotting with full-length HPV-5 genomic probe (arrow).

FIG. 4. Establishment of DNA replication from the cutaneous type of HPV-8 genome. The religated circular plasmid DNA of HPV-8 genome was titrated (2, 5, 10 μg) into U2OS cells. The linear 8 kb bands of the replicated episomal DNA (BamHIH/DpnI treated Hirt lysis samples) of HPV-8 genome, increasing in time and concentration dependently, are indicated by arrow.

FIG. 5-FIG. 12. Stable maintenance of HPV genomes in U2OS cells.

FIG. 5. Stable DNA replication of high- and low-risk HPV plasmids in U2OS cells. 5 μg of religated circular plasmid of HPV-6b, -11, -16, -18 together with 5 μg of AraD carrier DNA and with 2 μg of Eco0109I-linearized pNeo-EGFP plasmid were introduced into U2OS cells. The cells were put under G418 selection 48 h after the transfection and were grown with selection about three weeks post-transfection. Low-molecular-weight extrachromosomal DNA samples from parental cell pools, extracted by Hirt method, were analysed. Samples were digested with linearizing enzyme and HPV signals were detected by Southern blotting with mixed radiolabelled HPV probes. DNA samples, which were cultivated 3 weeks without G418 selection post transfection, are also shown.

FIG. 6-FIG. 12. Southern blot analysis of single cell subclones of different HPV subtypes in U2OS cell line. 5 μg of religated circular HPV plasmid together with 5 μg of carrier DNA (AraD) and 2 μg of linearized pNeo-EGFP or pBabeNeo plasmid was introduced into U2OS cells. Starting from 48 hrs after the transfection G418 selection was performed for about three weeks. Dilutions of 5000, 10 000 and 50 000 cells per 100 mm dish from the parental cell pools were transferred and single cell colonies were isolated, grown and analyzed. Total genomic DNA was isolated by standard method. 10 μg of linearized version of total cellular DNA was loaded on a gel and analyzed by Southern blotting with appropriate radiolabelled HPV genome-specific probe. Copy number was estimated by standard curves of marker lanes. Cell banks of these cell clones were generated.

FIG. 6. Series of HR-HPV18 positive U2OS cell lines containing stable HPV-18 plasmids at different levels. 10 μg of EcoRI-linearized total cellular DNA was analyzed by Southern blotting with radiolabelled full-length HPV-18 genome-specific probe. Clone numbers are indicated in the figure above the series and calculated copy numbers are shown by marker lanes. The identified cell clones carry different number of HPV-18 copies per cell.

FIG. 7. Analysis of HR-HPV16 positive clonal cell populations. 10 μg of BamHI-linearized total cellular DNA was analyzed by Southern blotting with radiolabelled full-length HPV-16 genome-specific probe. Calculated copy numbers and clone numbers are indicated in the figure. The identified cell clones carry different number of HPV-16 copies per cell, varying from low to high copy number.

FIG. 8. Series of LR-HPV11 positive U2OS cell lines containing stable HPV-11 plasmids at different level of content. 10 μg of BamHI-linearized total cellular DNA was analyzed by Southern blotting with radiolabelled full-length HPV-11 genome-specific probe. Calculated copy numbers and clone numbers are indicated in the figure. The identified cell clones carry different number of HPV-11 copies per cell.

FIG. 9. Series of LR-HPV6b positive U2OS cell lines containing stable HPV-6b plasmids at different levels. 10 μg of BamHI-linearized total cellular DNA was analyzed by Southern blotting with radiolabelled full-length HPV-6b genome-specific probe. Calculated copy numbers and clone numbers are indicated in the figure. The identified cell clones carry different number of HPV-6b copies per cell.

FIG. 10. Human U2OS cell lines with low to high number of copies of stable HPV-5 plasmids. 10 μg of SacI-linearized total cellular DNA was analyzed by Southern blotting with radiolabelled full-length HPV-5 genome-specific probe. Calculated copy numbers and clone numbers are indicated in the figure. The identified cell clones carry different number of cutaneous type of HPV-5 copies per cell.

FIG. 11. Human U2OS cell lines carrying low to high number of copies of stable HPV-8 plasmids per cell. BamHI-linearized total cellular DNA was analyzed by Southern blotting with radiolabelled full-length HPV-8 genome-specific probe. Calculated copy numbers and clone numbers are indicated in the figure. The identified cell clones carry different numbers of cutaneous type of HPV-8 copies per cell.

FIG. 12. Maintenance of HPV-18 genome in U2OS cell line. HPV-18 #1.13 subclone was cultivated in regular monolayer cell culture conditions during next 11 weeks after the first detection of the positivity of HPV-18 signal. Stability of extrachromosomal HPV-18 DNA over the time course was determined by Southern blot analysis of linearized low-molecular weight DNA samples from Hirt lysates extracted from 100 mm culture dish. In parallel 2 μg of linearized total cellular DNA was loaded and HPV-18 maintenance signal compared during the same time course.

FIG. 13-FIG. 18. The induction of DNA amplification demonstrated by the HPV-18 positive cell line U18 #1.13.

A sample from U18 #1.13 cell line was taken from the cell bank, cells were grown as regular monolayers, and 10⁶ cells were seeded into each of the six 100 mm culture dishes for additional cultivation. 2 ml of fresh culture medium (IMDM) was added every two days, but no splitting of the cells was performed. Time points for analysis were taken the next day after adding the medium during 12 day growth period with 2-days interval. Time dependent growth series to obtain dense cell cultures are presented.

FIG. 13. The growth curves of untransfected U2OS and HPV-18 positive cell line U18 #1.13.

Time dependent growth series to obtain dense cell cultures are presented. The cells were counted with Invitrogen Countess cell counter before analysis.

FIG. 14. Amount of summarized total DNA in time series.

Total DNA was isolated by standard procedures, and DNA concentrations were measured by NanoDrop spectrophotometer ND-1000.

FIG. 15. Southern blot analysis of the constant amount of total cellular DNA at different time points. Equal amounts (shown 10 μg) of total cellular DNA were digested with linearizing enzyme EcoRI, and the amplification of HPV-18 genome was detected with radiolabelled HPV-18 genome-specific probe. The induction of DNA amplification is demonstrated.

FIG. 16. Calculated HPV-18 copy numbers at different time points.

The replication signal intensities of U18 #1.13 cell line were measured using Phosphor-Imager and ImageQuant software. The HPV-18 genome copy number was estimated by standard curves of marker lanes. Three different series are summarized.

FIG. 17. RT-PCR analysis of U18 #1.13 cell line mRNA levels at different time points. mRNA levels of viral proteins were investigated at different time points during the induction of amplification. Total RNA was extracted with TRIzol reagent (Invitrogen) according to the manufacturer's protocol, and treated with DNase I (Fermentas) followed by heat inactivation of the enzyme. cDNA was synthesized with First Strand cDNA Synthesis kit (Fermentas) using 1 μg of total RNA as a template and oligo-dT primers in 20 μl reaction volume. cDNA was diluted into 160 μl and 2.5 μl of the dilution were used in a single PCR reaction along with 300 nM forward and reverse primers and 2 μl commercial master mix 5×HOT FIREPo1® EvaGreen® qPCR Mix (Solis Biodyne) in 10 μl of total reaction volume. Amplification was performed on 7900HT Real-Time PCR System (Applied Biosystems) and analyzed using comparative Ct (ΔCt) method, comparing HPV transcripts specific signals against reference gene β-actin signal. Signals were normalized to time point zero. RT-PCR analysis shows upregulation of the mRNA levels encoding viral proteins E1, E2, E6, E7, L1.

FIG. 18. The neutral/neutral two-dimensional gel analysis (N/N 2D) for determining the structure of DNA replication intermediates (RIs). The total DNA from U18 #1.13 cells grown as dense monolayer culture was analysed by digestion with HindIII enzyme as non-cutter for HPV-18 DNA, and separated on 2D gel. The sample of 10 μg of total DNA was loaded on a 0.4% agarose gel in 0.5×TBE buffer. The first dimension was electrophoresed at 10V for 48 hrs. The lane of interest was excised from the first dimension and rotated by 90°. 1% agarose gel in 0.5×TBE was run in the second dimension with EtBr (0.33 μg/ml) at 150V for 6 hrs. The DNA was transferred from the gel to a nylon filter, and probed with HPV-18 genome-specific probe. The size markers of superc oiled DNAs are shown in both directions. The presence of 8 kb circular plasmid is shown by arrow; the generation of high-molecular-weight plasmid multimers is also detected.

FIG. 19-FIG. 20. Increase in HPV-18 copy number in U2OS cells detected by fluorescence in situ hybridization. 10⁶ cells of U18 #1.13 cell line were seeded into 100 mm culture dish, and grown for 2 weeks in cell culture, adding 2 ml of fresh culture medium in every two days, but no splitting of the cells was performed. Samples were collected on the first and on the 14th day after seeding, and analyzed by fluorescence in situ hybridization (FISH) (Invitrogen Corporation, TSA™ Kit #22). Hybridization probes were generated by nick translation, using HPV-18 genome as template and biotin-16-dUTP as label. Cell nuclei were counterstained with DAPI and mounted in PBS with 50% glycerol.

FIG. 19. U18 #1.13 cells with HPV-18 signal on the first day after seeding.

FIG. 20. U18 #1.13 cells with HPV-18 signal 2 weeks after seeding. The HPV-18 positive signal has increased in dense cell culture due to the amplification of viral genomes.

FIG. 21. The plasmid pUCHPV-18E, (SEQ ID NO: 1)

Most of the late region (L1 and L2 ORFs) of the HPV-18 genome was removed by cleavage with ApaI and BpiI. The removed region was replaced with the fragment containing the sequences needed for propagation of the plasmid in E. coli cells (pMB1 origin of replication and beta-lactamase resistance markergene (bla) amplified from pUC18 cloning vector). The inserted bacterial sequences can be removed by HindIII digestion.

FIG. 22. The plasmid pUCHPV-18E-Gluc (SEQ ID NO: 2)

Expression cassette that includes synthetic 5′ intron element, codon optimised sequence encoding Gaussia luciferase marker gene, as well as bovine growth hormone polyadenylation signal, were inserted into the pUCHPV-18E so that the early region of the HPV-18 genome remained intact. The bacterial sequences can be removed by HindIII digestion.

FIG. 23. The plasmid pUCHPV-18E-TKGluc (SEQ ID NO: 3) The plasmid was made from the pUCHPV-18E-Gluc by insertion of the Herpes Simplex virus 1 (HSV 1) derived thymidine kinase (TK) promoter region in front of the Int-Gluc-bgh expression cassette. The bacterial sequences can be removed by HindIII digestion.

FIG. 24-27 New generation of plasmids

FIG. 24. Schematic maps of the markergenomes 18L2-Rluc and 18L2-RlucpA.

FIG. 25. Schematic map of the markergenome 18-E1-Rluc-E2. Scheme for expression and processing of the fusion polypeptdide that consists of first 24 aa of the E2, Rluc, 2A peptide and full-length E2 protein (E2′-Rluc-2A-E2) is indicated.

FIG. 26. Southern blot analysis of markergenomes replication in U2OS-EGFP-Fluc cells. The low molecular weight DNA was isolated and HPV18 or markergenome replication was analysed 48 and 72 hours post-transfection using DpnI assay and Southern blotting.

FIG. 27. Luciferase expression analysis from the markergenomes in U2OS-EGFP-Fluc cells. The cells were lysed 48 and 72 hours post-transfection and activities of firefly luciferase (indicated on top left, expressed by U2OS-EGFP-Fluc cells) and Renilla luciferase (indicated on top right, expressed by markergenomes) were measured. The firefly/Renilla ratios were calculated by data (indicated on bottom).

### DETAILED DESCRIPTION OF THE INVENTION

### Example 1

### Transient HPV DNA Replication in U2OS Cells

Human papillomaviruses show strong tropism for epithelial cells. It was discovered that human osteosarcoma cell line U2OS, exhibiting epithelial adherent morphology, although derived from a moderately differentiated osteosarcoma, supported very effectively the HPV E1 and E2 protein dependent viral DNA replication, when the expression-vectors for viral replication proteins were used together with reporter plasmids containing viral origin. U2OS cells encode wild-type pRb and p53.

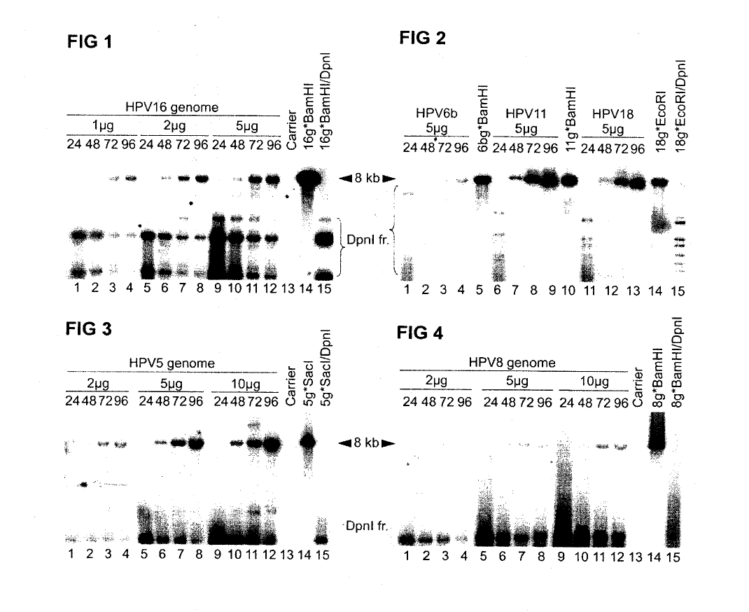

Hereafter it was investigated, whether the viral trans factors (E1 and E2) could act in their native configurations supporting the replication of the viral genomes in U2OS monolayer cultures. A set of four different cutaneous type of papillomaviruses were included, two of them belonging to high-risk type (HR/HPV-18 and HR/HPV-16) and two to low-risk type (LR/HPV-11 and LR/HPV-6b) according to their prognosis for cancer development. Additionally, two subtypes, the HPV-5 and HPV-8 as skin infecting β-papillomaviruses, were included. The U2OS cells were transfected with HPV-16 genome (FIG. 1), with HPV-6b, HPV-11, HPV-18 genomes (FIG. 2); with HPV-5 and HPV-8 genomes (FIG. 3 and FIG. 4, respectively) together with the carrier DNA (5 μg of AraD plasmid) and short term replication assay was performed. Prior to transfection, the HPV DNAs were cleaved out from the vector backbone: HPV-18 genome from pBR322 vector with EcoRI; HPV-6b from pBR322 with BamHI; HPV-16 and HPV-11 genomes from pUC19 with BamHI; HPV-8 DNA from pUC9 vector with BamHI; HPV-5 from pBR322 with Sad. Linear HPV fragments (ca 8 kb) were gel-purified and religated at low DNA concentrations in the ligation mix (30 μg/ml) for 16 hrs at 4° C.

As seen in FIG. 1, the introduction of increasing amounts (1, 2 and 5 μg) of the HPV-16 plasmid into the U2OS cells raises the viral DNA replication signal up with increase in time (FIG. 1, lanes 1-4, 5-8, 9-12) and in concentration-dependent fashion (FIG. 1, blocks of lanes 1-4; 5-8; 9-12). The same type short term transient replication pattern has been obtained in case of five other studied HPV types. As seen from the figures, the intensity of the linear 8 kb bands in the DpnI-treated samples (indicated by arrows) increases in time, which is considered as the indication of replication of viral genome in these cells (FIG. 2, lanes 1-4 in case of 5 μg of inserted HPV-6b plasmid DNA, lanes 7-10 with HPV-11 and lanes 11-14 with HPV-18 DNA and FIGS. 3 and 1D for HPV-5 and HPV-8, respectively). All transfected HPV plasmids can initiate viral DNA replication in the U2OS cell line at quite comparable levels in short-term assays as has been observed in independent experiments.

The fact that the diverse groups of HPV circular genomes of HPV-6b, HPV-11, HPV-16, HPV-18, HPV-5 and HPV-8, respectively, are capable of establishing viral DNA replication in U2OS cells, suggests that the viral regulatory elements are adequately functional for supporting DNA replication of these virus types and that viral and cellular transcription and replication factors are adequately expressed. Thus, a compound capable for the inhibition of the first amplificational step of viral DNA replication in U2OS cell culture may be considered as a potential candidate for HPV treatment/prevention of HPV infection. The observation is valid at least for high-risk and low-risk mucosal HPVs as well as cutaneous HPVs.

### Example 2

### HPV Stable Replication in U2OS Monolayer Cultures

### Establishment of Persistent HPV Stable Maintenance in U2OS Cell Line

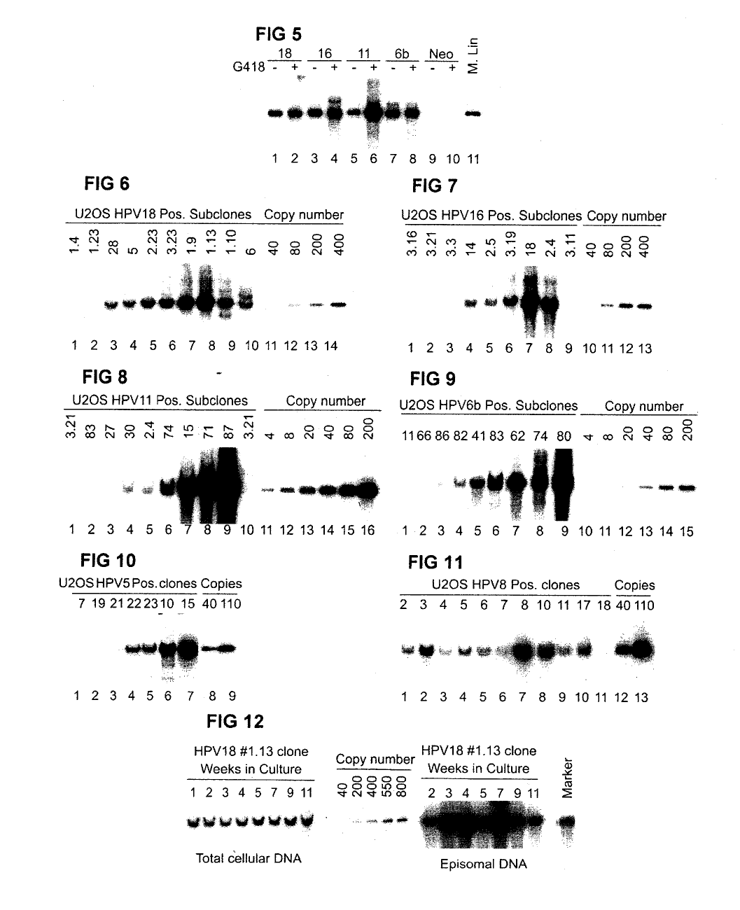

Quite strong HPV genomic DNA replication signal in U2OS cells in transient assays suggested further evaluation of the capacity of HR- and LR-HPV plasmids for stable episomal replication. For this purpose we co-transfected into U2OS cells 5 μg of HPV-6b, or HPV-11, HPV-16, HPV-18, HPV-5, HPV-8 circular plasmid together with 5 μg AraD carrier DNA and with 2 μg of Eco01091-linearized of pNeo-EGFP or EcoRI-linearized pBabeNeo plasmid, encoding antibiotic resistance marker, which would allow the selection for the transfected cells. 48 hrs after the transfection G418 selection was performed. After two to three weeks of cultivation with G418 selection, the low-molecular weight (LMW) Hirt extracts from whole cell population (“pool” DNA) were analyzed by Southern blotting with radioactively labelled probes against the appropriate HPV types. The analysis shows that all tested samples contained HPV genomes at quite comparable levels, which indicates that the selected cells contained the HPV replicon (FIG. 5). The transfected HPV genomes were quite efficiently maintained even in series without selection (FIG. 1).

For the detection of cloned human cell lines that carry extrachromosomal replicating HPV episomes, dilutions of 5000, 10 000 and 50 000 cells per 100 mm dish were transferred from selected cell population and the single cell colonies were picked, expanded, and grown up under the G418 selection. Total genomic DNA was extracted from these clones and Southern blot analysis was performed with 10 μg of EcoRI-linearized (FIG. 6), BamHI-linearized (FIG. 7, 8, 9, 11) or SacI-linearized (FIG. 10) total cellular DNA using appropriate radiolabelled full-length HPV subtype-specific probes. Sets of single cell subclones for every different HPV type in U2OS cell line were detected (FIG. 6-FIG. 11) and put into cell bank. In FIG. 6 and FIG. 7 positive examples of subclones of high-risk type of HPV-18 and HPV-16 are shown, carrying different copy numbers of the HPV genomes per cell line. The U2OS cell clones carrying low-risk type of HPV-11 and HPV-6 were also isolated (shown in FIG. 8 and FIG. 9) as well as the subclones for β-papillomavirus types HPV-5 and HPV-8 (shown in FIG. 10 and FIG. 11). The viral DNA copy number in different cell lines varied from very low to very high-copy per clone as indicated by Southern blotting. The copy number of the viral genomes was estimated using known quantities of the HPV plasmids on the same gel. Analysis of the episomal state of DNA plus FISH inspection was performed.

### Long Term Follow Up of HPV-Positive Subclones by Southern Blot Analysis

For isolated HPV-positive subclones long term follow up was performed by Southern blot analysis to determine the stability of the episomal maintenance replication continuing into later passages. The majority of the tested cell lines were stable in monolayer cultures with regular cultivation conditions in monolayer cultures during at least two months of inspection (example with HPV-18 subclone #1.13 on FIG. 12). A certain loss of plasmids existed in low-risk type of HPV-11 and HPV-6b inspection, if continuous passage of the cell-lines took place.

HPV-18 #1.13 subclone was cultivated in regular monolayer cell culture conditions during 11 weeks starting from the detection of positive HPV-18 signal. The stability of extrachromosomal HPV-18 DNA over the time course was determined by Southern blot analysis of linearized (EcoRI) low-molecular weight DNA samples from Hirt lysates, extracted every time from one 100 mm culture dish. In parallel series equal amount (2 μg) of linearized cellular DNA (total DNA) was loaded and compared during the same time course. The HPV-18 full length genome specific probe was used.

The fact that the diverse group of HPV circular genomes of HPV-6b, HPV-11, HPV-16, HPV-18, HPV-5 and HPV-8, respectively, are capable of maintaining viral DNA replication in U2OS cells in monolayer cultures, further suggests that the viral regulatory elements are adequately functional for supporting at least stable or latent viral DNA replication step of these virus types and that viral and cellular transcription and replication factors are adequately expressed. Thus, a compound capable for the inhibition of the latent step of DNA replication in U2OS cell culture may be considered a potential candidate for HPV treatment/prevention in the latent phase of HPV infection. The observation is valid at least for high-risk and low-risk mucosal HPVs as well as cutaneous HPVs. The establishment of subclones with the HPV plasmid copy numbers varying from low to high confirms the usefulness of created tools, desired in the search for anti-HPV drugs.

### Example 3

### Late Amplification of the HPV Genomes

### Genome Amplification in a Manner Similar to Differentiation-Dependent Viral Amplification

In the productive stage of PV life cycle, amplification of the viral genome occurs in differentiated cells within the upper layer of epidermis. To study the productive stage of viral life cycle in tissue culture, the three-dimensional architecture of the epithelium has been usually tried to be reproduced with organotypic or raft cultures, suspension in methylcellulose, feeder cells, by using regulated culture and growth conditions.

We used an alternative method, only dense cell cultures to imitate differentiation-dependent viral amplification. For this purpose equal number of cells (for example 1×10⁶ cells per 10 cm culture dish) of appropriate HPV-positive cell clone were split on several dishes (for example 6) and maintained as regular confluent monolayers grown up to high densities. The total DNA or low molecular weight (Hirt) DNA samples were collected at day 2, 4, 6, 8, 10, 12, ( . . . ), isolated and analyzed.

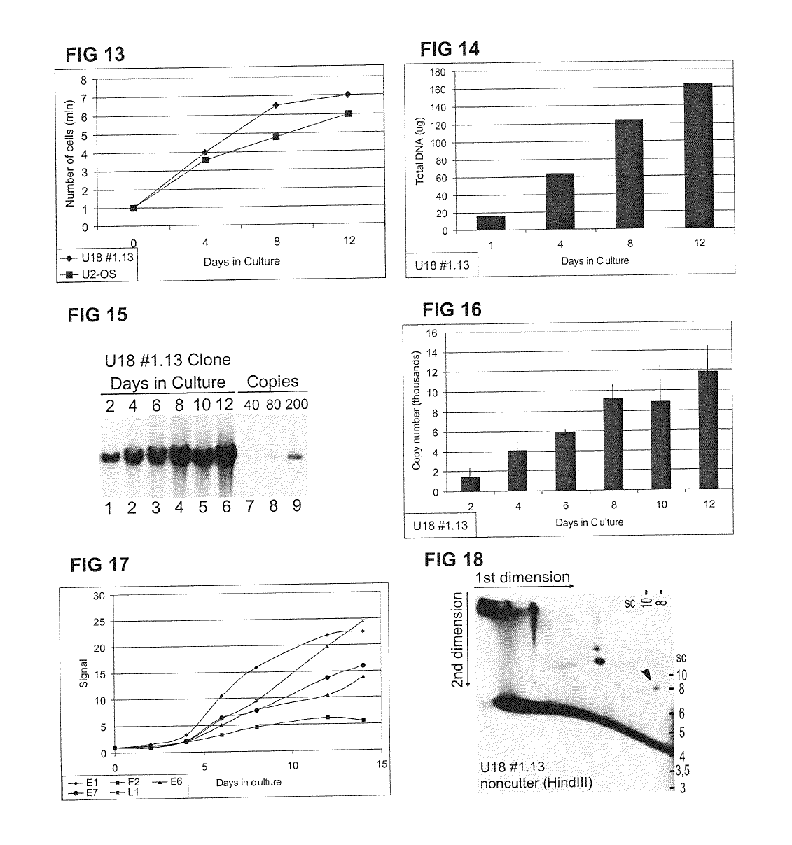

Using the HPV-18 positive cell line H18 #1.13 as an example, the induction of HPV DNA amplification is shown in FIG. 13-18. The same type of amplification was tested and observed in all HPV types under investigation including low risk as well as cutaneous beta HPV. Vegetative amplification was observed in all HPV-types. The examples of cell growth curves are given in FIG. 13 and the increasing amounts of total DNA extracted in series in FIG. 14. In FIG. 15, constant, equal amounts of total DNA from the series were loaded on the gel and analysed by Southern blot using EcoRI as a single cutter enzyme for HPV-18 and virus specific probe. The HPV DNA amplifies up at dense culture conditions (FIG. 15), shown in FIG. 16 with the quantitative data. Several repeated experiments were performed. RT-PCR analysis shows upregulation of synthesis of viral protein E1, E2, E6, E7, L1 RNA levels (FIG. 17). The neutral/neutral two-dimensional gel electrophoresis (2D) hybridization pattern indicates the presence of monomeric and multimeric forms of HPV-plasmids (FIG. 18). The differences in the shape of DNA replication intermediates in 2D restriction analysis at two stages (first and 12. day after seeding) would be the indication that the replication mode has been changed.

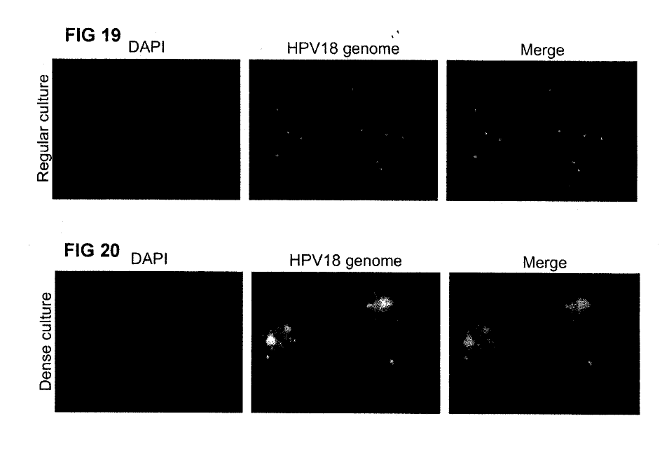

To characterize the appearance of intracellular HPV DNA episome formation supplementary to Southern blot analysis, the interphase and metaphase fluorescence in situ hybridization (FISH) was performed for studied subclones (Invitrogen Corporation, TSA™ Kit #22). Examples for interphase FISH for HPV-18 subclone #1.13 are shown in FIG. 19-FIG. 20. The U18 #1.13 cells exhibit HPV-18 signal on the first day after seeding (FIG. 19). Two weeks after seeding the HPV-18 positive signal in U18 #1.13 cells has increased due to the amplification of viral genomes (FIG. 20).

As seen from these examples, HPV plasmid goes through an amplificational replication stage in confluent U2OS cells, bringing its copy number up to tens of thousands per cell, and therefore it is applicable for a person skilled in the art to use it in a high-throughput system for screening for agents exhibiting anti-HPV properties. Thus, a compound capable for the inhibition of amplificational DNA replication in U2OS cell culture may be considered a potential candidate for HPV treatment/prevention in the amplificational phase of HPV infection. The observation is valid at least for high-risk and low-risk mucosal HPVs as well as cutaneous HPVs.

### Example 4

### The Plasmid pUCHPV-18E, (SEQ ID NO:1)

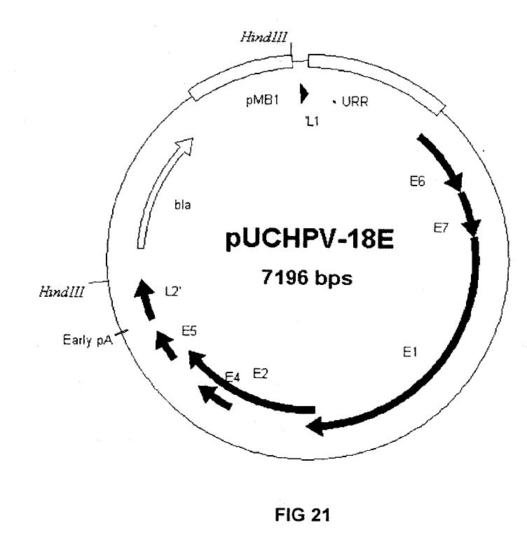

Most of the late region (L1 and L2 ORFs) of the HPV-18 genome was removed by cleavage with ApaI and BpiI. The removed region was replaced with the fragment containing the sequences needed for the propagation of the plasmid in E. coli cells (pMB1 origin of replication and beta-lactamase resistance marker gene (bla) amplified from pUC18 cloning vector). The inserted bacterial sequences were removed by HindIII digestion. As a result, a plasmid construct with HPV-18 early region was obtained. The map of the plasmid is presented in FIG. 21.

### Example 5

### The plasmid pUCHPV-18E-Gluc (SEQ ID NO:2)

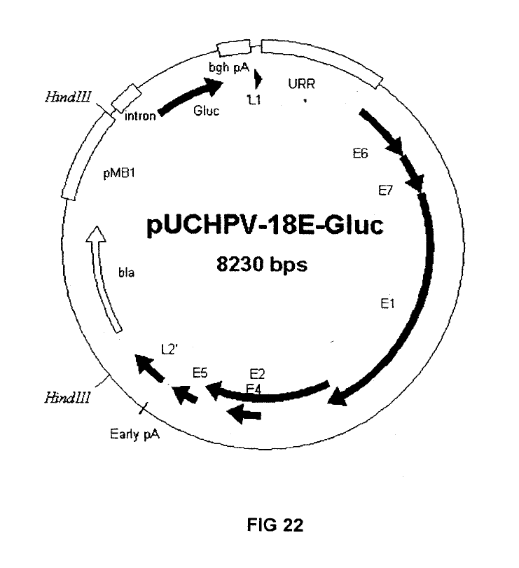

Expression cassette that includes synthetic 5′ intron element, codon optimised sequence encoding Gaussia luciferase marker gene, as well as bovine growth hormone polyadenylation signal, were inserted into the pUCHPV-18E so that the early region of the HPV-18 genome remained intact. The bacterial sequences were removed by HindIII digestion. As a result, a plasmid with HPV-18 early region was constructed, which carries a reporter gene enabling quantitative or semi-quantitative detection of extrachromosomal high-risk mucosal HPV-18 DNA. The map of the plasmid is presented in FIG. 22.

### Example 6

### The Plasmid pUCHPV-18E-TKGluc (SEQ ID NO 3)

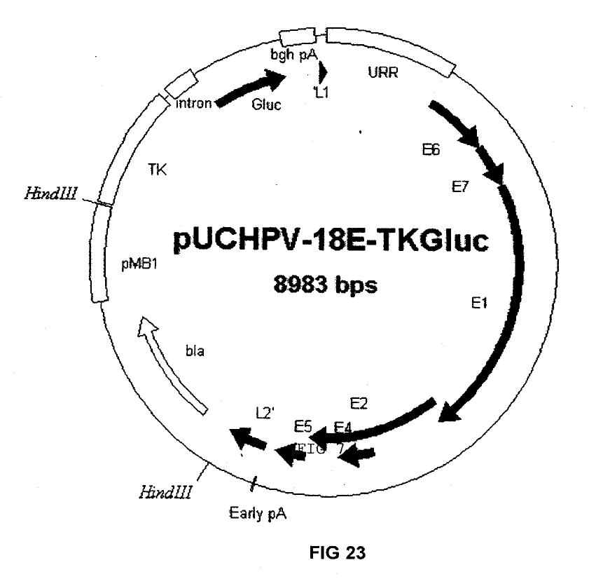

The plasmid was made from the pUCHPV-18E-Gluc by insertion of the Herpes Simplex virus 1 (HSV 1) derived thymidine kinase (TK) promoter region in front of the Int-Gluc-bgh expression cassette. The bacterial sequences were removed by HindIII digestion. As a result, a plasmid with HPV-18 early region was constructed, which carries a TK promoter-regulated reporter gene enabling quantitative or semi-quantitative detection of extrachromosomal high-risk mucosal HPV-18 DNA. The map of the plasmid is presented in FIG. 23.

Examples 4-6 present a HPV-based construct, where L1 and L2 genes have been removed and replaced with a reporter gene. Accordingly, a useful instrument for quantitative or semi-quantitative assessment of the amount of replicated extrachromosomal DNA is provided.

### Example 7

### The Plasmids pMC-18L2-Rluc (SEQ ID NO:4) and pMC-18L2-Rluc-pA (SEQ ID NO:5)

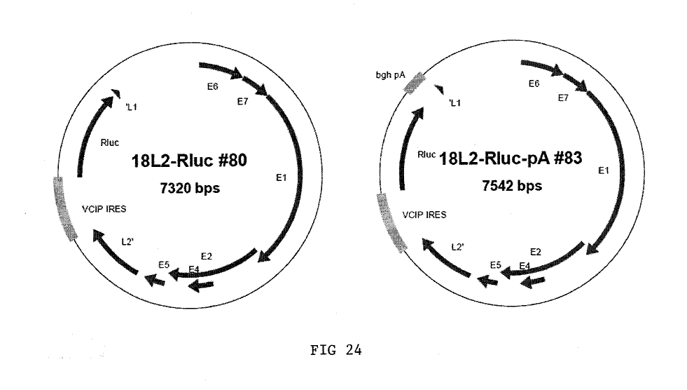

Constructs pMC-18L2-Rluc (SEQ ID NO:4) and pMC-18L2-Rluc-pA(SEQ ID NO:5) were cloned as parental plasmids for preparation of HPV18 markergenomes 18L2-Rluc (SEQ ID NO:6) and 18L2-Rluc-pA (SEQ ID NO:7), respectively (FIG. 24). The markergenomes are usable tools for HPV replication inhibition studies by analysing the viral copy number by expression level of markergene. The pMC (pMC.BESBX) backbone used for cloning is described previously and it allows the purification of inserted markergenomes as minicircle plasmids from which the bacterial backbone sequences are removed during propagation in E. coli cells.

The markergenomes were constructed by inserting markergene (Renilla luciferase (Rluc) in this particular example into the late region (L1 and L2 ORFs) of HPV18 genome downstream from the sequences needed for polyadenylation of the viral early transcripts. As cellular transcription factor binding sites containing heterologous promoter sequences could interfere the HPV gene we did not include any promoter into the markergene expression cassette. Instead of this, the Rluc cDNA was linked with human VCIP mRNA 5′UTR for promotion of the markergene expression. It has been demonstrated that VCIP mRNA 5′UTR contains internal ribosome entry site (IRES) functional in U2OS cells (Blais et al., 2006).

pMC18L2-Rluc

First, the VCIP mRNA 5′UTR product was amplified from genomic DNA of U2OS cells using primers VCIP_F_PpuMI (SEQ ID NO: 8) and VCIP_R_MCS (SEQ ID NO:9). The VCIP mRNA 5′UTR and Rluc cDNA (derived from Rluc expression vector as NcoI-NotI fragment) was joined in cloning vector pTZ57R/T (Fermentas, Lithuania) resulting pTZ-VCIP-Rluc. For generation of the pMC18L2-Rluc (SEQ ID NO:4), the VCIP-Rluc fragment (cut out with PpuMI and Esp3I from pTZ-VCIP-Rluc) was inserted into the parental plasmid pMC-HPV18 for wt HPV18 opened with restriction enzymes PpuMI and BbsI.

pMC18L2-Rluc-pA

Rluc cDNA with 3′-linked bovine growth hormone gene polyadenylation region (pA) (derived from Rluc expression vector as NcoI-PacI fragment) was joined with VCIP mRNA 5′UTR product in the cloning vector pTZ57R/T (Fermentas, Lithuania) resulting the plasmid TZ-VCIP-Rluc-pA. For generation of the pMC18L2-Rluc-pA (SEQ ID NO:5), the VCIP-Rluc-pA fragment (cut out with PpuMI and Esp3I) was inserted into the plasmid pMC-HPV18 opened with restriction enzymes PpuMI and BbsI.

### Example 8

### The Plasmid pMC18-E1-Rluc-E2 (SEQ ID NO:10)

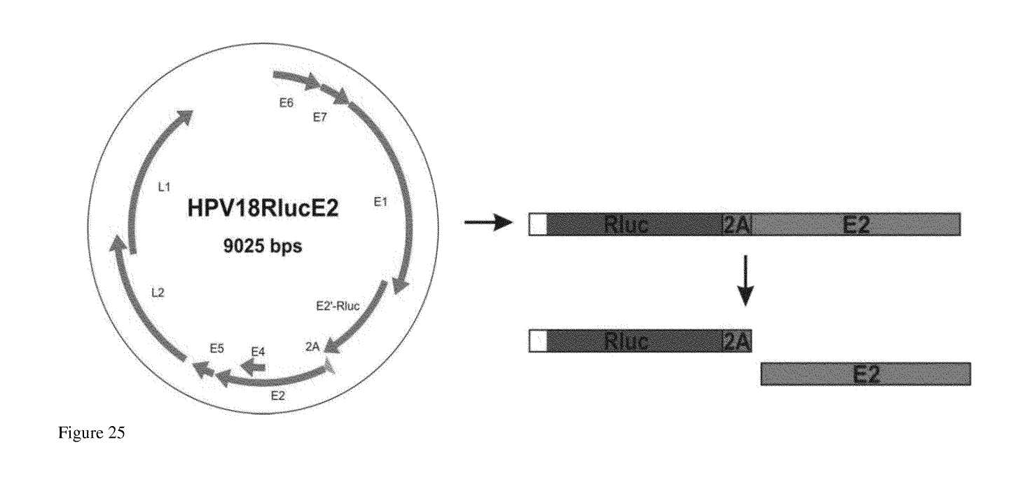

We also constructed the parental plasmid pMC18-E1-Rluc-E2 (SEQ ID NO:10) of another type of the markergenome, 18-E1-Rluc-E2 (SEQ ID NO: 11) (FIG. 25). In this conformation the markergene (Rluc in particular example), was inserted into the early region of the viral genome and no heterologous transcription regulatory sequences (promoter or polyadenylation signal) were included. In particular, the Rluc was inserted between E1 and E2 ORFs encoding the viral replication proteins E1 and E2, respectively. As the 3′ end of the E1 cDNA and 5′ end of the E2 cDNA are overlapping (71 nt), the Rluc cDNA was inserted without ATG start codon. Instead, the translation is started from native start codon of the E2 ORF and the Rluc is expressed as fusion protein with 24 N-terminal amino acids of the E2 protein which are encoded with the overlapping region. In addition, foot and mouth disease virus (FMDV) derived 2A peptide (24 aa) and full-length E2 cDNA coding sequences were fused in-frame with the 3′-end of the Rluc cDNA. The FMDV 2A peptide initiates the co-translational “cleavage” of the nascent polypeptide into two separate proteins. Thus, by this configuration the translation of viral E2 encoding mRNAs initiated from E2 native start codon produces the fusion polypeptdide that consists of first 24 aa of the E2, Rluc, 2A peptide and full-length E2 protein (E2′-Rluc-2A-E2). The polypeptide is co-translationally processed by 2A directed mode to final products: E2′-Rluc-2A markergene and E2 (contains N-terminal single proline derived from 2A peptide), see FIG. 25.

The constructions were made as follows: Rluc cDNA fused with 5′ nucleotides of E2 ORF (including the Psp1406I site in E2 ORF) and 3′ part of the 2A peptide coding sequence was amplified from Rluc expression vector using primers E2-Rluc_F_Psp1406 and Rluc2A_R_Eam (SEQ ID NO:12 and 13, respectively). Also, the 5′ end of the E2 ORF (including the AatII site in the E2 ORF) fused with 5′ part of the 2A peptide coding sequence was amplified from HPV18 genomic DNA using primers 2AE2—F_Eam and E2—R_AatII (SEQ ID NO: 14 and 15, respectively). The amplified fragments were joined in pUC57-kana cloning vector using the Eam1105I site present in 2A peptide coding sequence. Finally, the pMC18-E1-Rluc-E2 (SEQ ID NO: 10) was generated by insertion of the E2′-Rluc-2A-E2′ construction from pUC57 into the pMC-HPV18 using the Psp1406I and AatII cloning sites present in the E2 ORF.

### Example 9

### Replication Properties of the 18L2-Rluc, 18L2-Rluc-pA and 18-E1-Rluc-E2 Markergenomes in U2OS Cells

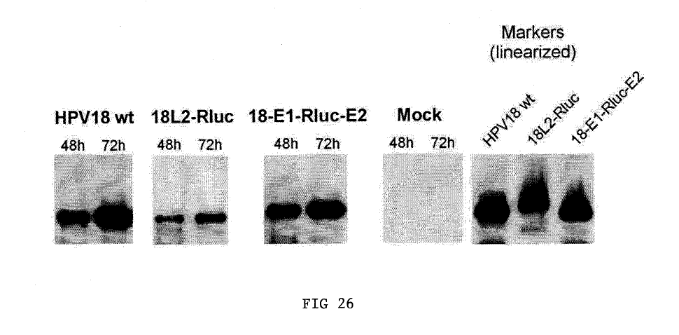

Transient replication assay was performed in U2OS cells in order to test the replication capability of the constructed markergenomes in comparison with wt HPV18 genome. First, the wt HPV18 genome and 18L2-Rluc, 18L2-Rluc-pA, 18-E1-Rluc-E2 markergenomes were prepared from their parental plasmids (pMC-HPV18, pMC-18L2-Rluc, pMC-18L2-Rluc-pA and pMC-18-E1-Rluc-E2, respectively) by removing almost completely the bacterial backbone sequences using the method described in Kay et al., 2010. Then the U2OS-EGFP-Fluc cells (U2OS derived cell line expressing EGFP and firefly luciferase) were transfected with 1 μg of HPV18 genome or 1 μg of each markergenome or mock transfected (neg. control). Forty-eight and 72 hours after transfection the low molecular weight DNA was isolated from the cells, digested with the restriction endonuclease linearizing the HPV18 genome or markergenomes and with DpnI (destroys the unreplicated input DNA). The digested DNA samples were analyzed by Southern blotting using early region of the HPV18 genome as the probe. The results shown on the FIG. 26 indicate that 18L2-Rluc, 18L2-Rluc-pA, 18-E1-Rluc-E2 can replicate in U2OS cells. The replication capability was higher for 18-E1-Rluc-E2 markergenome showed the replication levels similar to wt HPV18.

### Example 10

### Testing the Markergene Expression Properties of the 18L2-Rluc-pA and 18-E1-Rluc-E2 Markergenomes in U2OS Cells

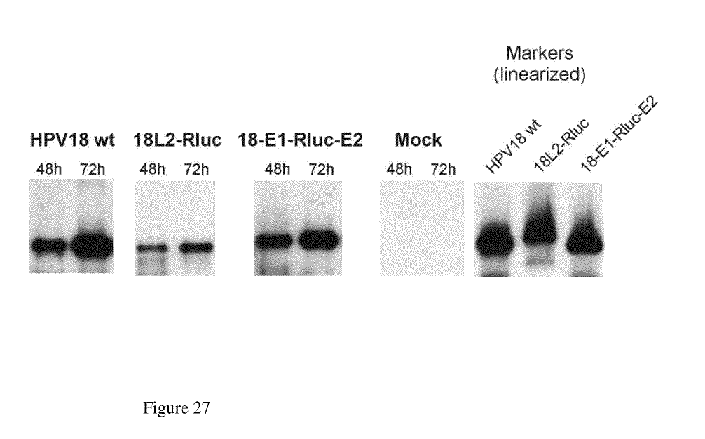

Similarly to replication assay described in Example 9 above, the markergene expression assay was performed in U2OS-EGFP-Fluc cells in order to test the markergene expression capability of the 18L2-Rluc-pA and 18-E1-Rluc-E2 markergenomes. The U2OS-EGFP-Fluc cells were transfected with 1 μg of HPV18 genome negative control (contains no markergene) or 1 μg of each markergenome. Forty-eight and 72 hours after transfection the cells were lysed and activities of firefly luciferase (expressed by U2OS-EGFP-Fluc cells) and Renilla luciferase (expressed by markergenomes) were measured in lysates using Dual-Luciferase® Reporter Assay System kit (Promega, US). The results shown in FIG. 27 indicate that Renilla luciferase markergene is expressed from 18L2-Rluc-pA and 18-E1-Rluc-E2 markergenomes.

U2OS cell lines transfected with plasmids of Examples 7 and 8 were tested for induction of vegetative amplification of HPV DNA similarly as described in Example 3. The results (not shown) prove that the cell lines are supporting all replications phases of HPV DNA including the vegetative amplification phase, and therefore are useful in establishing an in vitro system for high throughput screening for drugs inhbiting HPV DNA replication during any one of the replication phases.

### Example 11

### A Kit for Detecting Compounds Capable of Inhibiting HPV DNA Replication

A kit was completed by combining human osteosarcoma cell line U2OS, extrachromosomally maintainable HPV DNA plasmid pUCHPV-18E-TKGluc wherein the L1 and L2 genes are substituted with Gaussia luciferase marker gene. This construct was transfected into the U2OS cell line, the stable cell lines identified and cultivated to confluency. Any library of chemical compounds available or generated by a person skilled in the art can be applied to the preconfluent and/or confluent cell culture to screen the provided compounds from the library for their anti-HPV activity at stable maintenance and/or amplificational stage of viral DNA replication. The Gaussia luciferase reporter gene works as a means for quantitative or semi-quantitative assessment of replicated extrachromosomal DNA, as the amount of the fluorescent product of the inserted gene is readily detectable for a person skilled in the art either quantitatively by measuring the fluorescence or semi-quantitatively by visual observation with fluorescence microscope. Similarly a kit was completed by using U2OS cell line and extrachromosomally maintainable plasmids pMC-18L2Rluc, pMC-18L2Rluc-pA and pMC18-E1-Rluc-E2 The skilled artisan will recognise that instead of HPV 18-genome, genome from another type of human papilloma virus may be used.

### Example 12

### A Method for Identifying Compounds Capable of Inhibiting HPV DNA Replication

Complete or partial sequence of HPV DNA carrying all necessary viral cis-sequences and trans-factors necessary for all steps of viral replication cycles was introduced into human osteosarcoma cell line U2OS using electroporation or chemical transfection methods know in the art. The clones of U2OS cell lines that carry extrachromosomally replicating HPV plasmids was isolated using selection marker providing resistance to G418. The identified cell clones carrying different HPV copy numbers per cell were characterized, grown and cell banks of these cell clones were generated. The cells of the subclone chosen for the identification of HPV latent replication inhibitors were seeded at low density into 96 well plates, drug candidates at different concentrations were added to the growth media, and cells were grown until confluent. Alternatively cells may be seeded into 384 well plates to increase the throughput. As another, preferred option, the cell culture was maintained for at least 5 to 7 days on the plates to become confluent, the potential drug candidates were added to the growth medium after cells had become confluent. The copy-number of HPV extrachromosomal copies was determined in the cells by direct differential measurement of the viral DNA in the cells or using reporters. Subsequently, the compound under investigation was applied to the cultivation vessel of the U2OS cell clone monolayers; the presence or absence of the inhibitory effect of the compound on viral DNA stable or amplificational replication in the cells was assessed by measuring the amount of the product of the reporter gene or the amount of extrachromosomal DNA; finally the compound was identified as a candidate for HPV DNA replication inhibitor, if inhibitory effect on HPV DNA replication of a certain concentration of the compound at certain copy number level at certain growth phase is observed at certain growth conditions.

One skilled in the art will recognize that the examples above are illustrative and do not limit the scope of the invention. There are various ways of modifications that would fall under the spirit of this invention.

1. A method for identifying compounds capable of inhibiting Human Papilloma Virus (HPV) replication at initial replication phase, stable maintenance phase or at vegetative amplification phase, said method comprising the steps of:
a. introducing HPV genomic or subgenomic DNA into a human osteosarcoma U2SO cell line enabling initial replication, stable maintenance and vegetative amplificational replication of HPV DNA;
b. generating a collection of stable single cell subclones carrying extrachromosomal HPV DNA at different copy numbers per subclone;
c. cultivating cells of selected subclones as dispersed or dense monolayer cultures with regular media;
d. applying a compound under investigation to the monolayer of the subclone of cells carrying the HPV DNA;
e. assessing a presence or an absence of inhibitory effect of the compound on viral DNA maintenance or amplification in the cells;
wherein presence of inhibitory effect of the compound results in classification of the compound as a replication inhibitor candidate.

2. The method according to claim 1, wherein the compound under investigation is applied to the cell subclone monolayer before obtaining confluency; and the compound is tested for inhibition of latent phase of HPV DNA replication.

3. The method according to claim 1, wherein the culture of the subclone is maintained by consecutive passages at confluency for at least 4 to 12 days until vegetative amplificational replication phase of the extrachromosomal HPV DNA launches and the compound under investigation is applied to the medium of the cell subclone monolayer at confluency, and the compound is tested for inhibition of vegetative amplificational phase of HPV DNA replication.

4. The method according to claim 1, wherein the presence or absence of the inhibitory effect is assessed by measuring quantitatively or semi-quantitatively the amount of extrachromosomal viral DNA.

5. The method according to claim 1, wherein a sequence of a reporter gene is inserted to the subgenomic fragment of the HPV DNA.

6. The method of claim 5, wherein the sequence of the reporter gene substitutes L1 and L2 seqeunces of HPV.

7. The method of claim 5, wherein the sequence of the reporter gene is inserted in E2 ORF after E1 coding sequence.

8. The method of claim 5, wherein the reporter gene is d1GFP, luciferase, secreted alkaline phosphatase (SEAP), or Gaussia luciferase.

9. The method of claim 1, wherein the subgenomic fragment including the reporter gene sequence is introduced into the U2SO cell line by transfecting the cell line with a plasmid having nucleotide sequence according to SEQ ID NO:1, SEQ ID NO:2, SEQ ID NO:3, SEQ ID NO:4, SEQ ID NO:5 or SEQ ID NO:10.

10. The method of claim 5, wherein amount of a protein encoded by the reporter gene is measured.

11. The method of claim 5, wherein a product of reaction catalysed by a protein encoded by the reporter gene is measured.

12. The method according to claim 1, wherein the HPV is selected from a group consisting of high-risk mucosal HPV, low-risk mucosal HPV and cutaneous type of HPV.

13. The method of claim 12, wherein the HPV is a high-risk mucosal HPV selected from the group consisting of subtype HPV-18 and HPV-16.

14. The method of claim 12, wherein the HPV is low-risk mucosal HPV selected from the group consisting of subtype HPV-6b and HPV-11.

15. The method of claim 12, wherein the HPV is cutaneous type of HPV selected from the group consisting of subtype HPV-5 and HPV-8.

16. A compound capable of inhibiting Human Papilloma Virus (HPV) replication at initial replication phase, stable maintenance phase or at vegetative amplification phase, wherein said compound is identified according to method of claim 1.

17. A transfected human osteosarcoma cell line USO2 enabling initial replication, stable maintenance and vegetative amplificational replication of HPV DNA, said cell line carrying an extrachromosomally maintainable plasmid comprising a complete or partial HPV DNA sequence carrying all viral cis-sequences and trans-factors ensuring all steps of viral replication cycle and one or more reporter gene sequences.

18. The cell line of claim 17, wherein the plasmid is according to SEQ ID NO:1, SEQ ID NO:2, SEQ ID NO:3, SEQ ID NO:4, SEQ ID NO:5 or SEQ ID NO:10.

19. The transfected USO2 cell line of claim 17, wherein the cell line is U2OS-EGFP-Fluc.

20. An extrachromosomally maintainable plasmid for transfecting human osteosarcoma cell lines supporting all phases of HPV DNA replications, said plasmid comprising a complete or partial HPV DNA sequence carrying all viral cis-sequences and trans-factors ensuring all steps of viral replication cycle and one or more reporter gene sequences.

21. The plasmid of claim 20, wherein the reporter gene sequences substitute L1, L2 or both L1 and L2 sequences of viral genome.

22. The plasmid of claim 20, wherein the plasmid has nucleotide sequence according to SEQ ID NO:1, SEQ ID NO:2, SEQ ID NO:3 or SEQ ID NO:5.

23. The plasmid of claim 20, wherein the reporter gene sequences are inserted in E2 ORF after E1 coding sequence.

24. The plasmid of claim 23, wherein the plasmid is according to SEQ ID NO:10.

25. A kit for identifying compounds capable of inhibiting HPV replication at initial replication, stable maintenance or vegetative amplificational phase, said kit comprising:
a. human osteosarcoma cell line U2OS;
b. an extrachromosomally maintainable construct comprising a complete or partial HPV DNA sequence carrying all viral cis-sequences and trans-factors ensuring all steps of viral replication cycle and one or more reporter gene sequences for introduction into the U2OS cell line;
c. a compound or a library of compounds to be screened for anti-HPV activity;
d. a means for quantitative assessment of replicational, transcriptional or translational activity of HPV DNA in the cells.

26. The kit of claim 25, wherein the reporter gene sequences substitute viral L1 or L2 sequences or both of them.

27. The kit of claim 25, wherein the reporter gene sequence is inserted in E2 ORF after E1 coding sequence and a sequence comprising FMDV 2A coding sequence and full length E2 cDNA coding sequence is fused with 3′-end of the reporter gene sequence, and step d) comprises quantifying a fusion protein comprising partial E2-sequence and the protein encoded by the reporter gene sequences.

28. The kit of claim 25, wherein the extrachromosomally maintainable construct is according to SEQ ID NO:1, SEQ ID NO:2, SEQ ID NO:3, SEQ ID NO:4, SEQ ID NO:5 or SEQ ID NO:10.

29. An in vitro system for providing initial replication, stable maintenance and vegetative amplificational replication of HPV DNA, said system comprising a culture of human osteosarcoma cell line U2OS transfected with an extrachromosomally maintainable plasmid comprising a HPV DNA sequence carrying all viral cis-sequences and trans-factors ensuring all steps of viral replication cycle and one or more reporter gene sequences;
wherein said system is for high throughput screening of compounds inhibiting DNA replication at initial replication, stable maintenance or vegetative amplificational replication phase of low-risk, high-risk and skin-type of HPV.

30. The system of claim 29, wherein the reporter gene sequence substitute viral L1 or L2 sequence or both of them.

31. The system of claim 29 wherein the plasmid is according to SEQ ID NO:1, SEQ ID NO:2, SEQ ID NO:3, SEQ ID NO:4, SEQ ID NO:5 or SEQ ID NO:10.

32. The system of claim 29, wherein the reporter gene sequence is inserted in E2 ORF after E1 coding sequence and a sequence comprising FMDV 2A coding sequence and full length E2 cDNA coding sequence is fused with 3′-end of the reporter gene sequence, and inhibition of DNA replication is determined by monitoring changes in quantity of a fusion protein comprising partial E2-sequence and the protein encoded by the reporter gene sequences.

33. The system of claim 29 for use to screen compounds inhibiting late amplification replication of skin-type HPV for identification of compounds effective to prevent or cure viral infections in nondividing cells in upper layers of skin.
